(19) United States
(12) Patent Application Publication
(10) Pub. No.: US 2021/0186834 A1
(43) Pub. Date: Jun. 24, 2021

(54) TAHITI VANILLA EXTRACT AND 4-[(1E,3S)-3-ETHENYL-3,7-DIMETHYLOCTA-1,6-DIENYL]PHENOL FOR COMBATTING CUTANEOUS AGEING

(71) Applicant: PIERRE FABRE DERMO-COSMETIQUE, BOULOGNE-BILLANCOURT (FR)

(72) Inventors: Hélène DUPLAN, AUZEVILLE TOLOSAN (FR); Daniel BACQUEVILLE, TOULOUSE (FR); Stéphane POIGNY, SAUBENS (FR)

(73) Assignee: PIERRE FABRE DERMO-COSMETIQUE, BOULOGNE-BILLANCOURT (FR)

(21) Appl. No.: 17/057,578

(22) PCT Filed: May 23, 2019
(86) PCT No.: PCT/EP2019/063321

§ 371 Date: Nov. 20, 2020

(30) Foreign Application Priority Data
May 23, 2018 (FR) 1854294

(51) Int. Cl. A61K 8/34; A61Q 19/08; A61K 8/9783

(52) U.S. Cl. CPC A61K 8/347; A61Q 19/08; A61K 2800/26; A61K 2800/10; A61K 8/9783

(57) ABSTRACT

The present invention relates to a combination including 4-[(1E,3S)-3-ethenyl-3,7-dimethylocta-1,6-dienyl]phenol and a Tahiti vanilla extract, and the use of said combination for combatting cutaneous ageing.

### TECHNICAL FIELD

The present invention relates to the use of a combination of 4-[(1E,3S)-3-ethenyl-3,7-dimethylocta-1,6-dienyl]phenol and a Tahiti vanilla extract for combatting ageing of the skin, and cosmetic or dermatological compositions containing this combination intended to combat cutaneous ageing.

### PRIOR ART

The skin is the most extended organ of the human body, with nearly 2 m² of surface area. The skin owes its suppleness and its resistance to the different layers of tissues that compose it: the epidermis, the dermis and the hypodermis. The epidermis, the outer layer of the skin, is responsible for its impermeability and its resistance. It is essentially composed of keratin, a fibrous protein produced by keratinocytes, and melanin, the main cutaneous pigment produced by melanocytes. Over the years, the renewal of keratinocytes takes place more slowly and their terminal differentiation is slowed down. Over time, profound modifications occur at the level of the dermis. The support tissue of the skin, it is 80% constituted of elastin and collagen fibres immersed in a gel of glycoproteins. Fibroblasts, which are the main cells of the dermis, are specialised in the synthesis of these elastin and collagen fibres. They ensure equilibrium between synthesis, maturation and degradation of elastin and collagen fibres. Between 20 and 80 years, the fibroblast population is decreased by half. Thus, over time, this equilibrium is going to be displaced towards a degradation of these fibres with for result a loss of elasticity and tonicity of the dermis and a flaccidity which no longer opposes the effects of contraction of the underlying muscles, leading to the appearance of wrinkles. Collagen fibres are distributed in all the layers of the dermis, they are capable of fixing water and contribute to the moisturising of the skin. A decrease in collagen and/or a modification of the quality thereof leads to the appearance of deep wrinkles. With ageing, elastin fibres, which confer elasticity and solidity to the tissues, become rarer, the skin becomes thinner and wrinkles. The skin has to continually deal with multiple external aggressions which can accelerate the natural process of ageing. It is particularly sensitive to attacks of free radicals generated both by the normal functioning of our body and by external elements, such as pollution, tobacco, but above all solar radiation. These free radicals are responsible for tissue and cellular modifications that lead to cutaneous ageing.

After exposure to UV, there is an increase in the expression of metalloproteinases in the epidermis and the dermis. These enzymes that alter the production of collagen in the dermis contribute to the appearance of wrinkles. The induction of these enzymes is maintained by repeated exposures. There is a thickening of the dermis due to the accumulation of elastic material. The production of interleukins by the epidermis, following UV irradiation, contributes to the destruction of the conjunctive tissue. It would seem that the effects observed in the dermis are rather attributable to UVA, which penetrate deeper than UVB. In hairless mice, as in humans, the appearance of hyperplasia and wrinkles are observed after irradiation with wavelengths comprised between 295 and 300 nm, the collagen fibres are damaged and there is production of abnormal elastic fibres: this is known as elastosis. After 10 weeks of irradiation with UVA, there are no longer any normal elastic fibres.

There still exists a need to have available new efficient active ingredients or combinations of active ingredients for combatting cutaneous ageing.

### SUMMARY OF THE INVENTION

Surprisingly, the Inventors have demonstrated a synergy for combatting the signs of cutaneous ageing by combining a Tahiti vanilla extract with 4-[(1E,3S)-3-ethenyl-3,7-dimethylocta-1,6-dienyl]phenol.

The aim of the present invention is thus to propose the use by topical route of a novel combination of active ingredients for combatting ageing of the skin.

The present invention thus relates to a combination including 4-[(1E,3S)-3-ethenyl-3,7-dimethylocta-1,6-dienyl]phenol and a Tahiti vanilla extract, more particularly a Tahiti vanilla pod extract.

The present invention also relates to a cosmetic or dermatological composition including, as active ingredient, a combination according to the invention, and at least one cosmetically or dermatologically acceptable vehicle.

The present invention also relates to a combination according to the invention or a composition according to the invention, for the topical use thereof for combatting cutaneous ageing, and/or preventing and/or reducing wrinkles, and/or toning up the skin, and/or relaunching epidermal and dermal cellular activity.

The present invention also relates to the cosmetic use of a combination according to the invention or a composition according to the invention, for combatting cutaneous ageing, and/or preventing and/or reducing wrinkles, and/or toning up the skin, and/or relaunching epidermal and dermal cellular activity.

### Definitions

The terms “4-[(1E,3S)-3-ethenyl-3,7-dimethylocta-1,6-dienyl]phenol” and “bakuchiol” are used in an interchangeable manner in the present description.

“Tahiti vanilla extract” is taken to designate the extraction product of ripe pods of Vanilla tahitensis.

“Extraction product” is taken to mean the product obtained after extraction of ripe pods of Vanilla tahitensis with a solvent, called extraction solvent, (that is to say a liquid solution in the extraction solvent) optionally in a concentrated or dry form after partial or total evaporation of the extraction solvent.

“Hydrophilic extract” is taken to mean an extract obtained by the use of at least one hydrophilic solvent as extraction solvent. Preferably, it is an extract obtained by extraction using one or more hydrophilic solvents.

“Aqueous extract” is taken to mean an extract obtained by the use of at least one aqueous solvent as extraction solvent. Preferably, it is an extract obtained by extraction using one or more aqueous solvents.

“Hydro-glycolic extract” is taken to mean an extract obtained by the use of at least one hydro-glycolic solvent as extraction solvent. Preferably, it is an extract obtained by extraction using one or more hydro-glycolic solvents.

“Hydrophilic solvent” is taken to mean a solvent selected from the group consisting of water, C1 to C5 alcohols (for example ethanol), C3 to C5 glycols (for example propylene glycol, butylene glycol, pentylene glycol, propan-1,3-diol), glycerol, acetone, C1 to C5 alkyl esters (for example ethyl acetate, isopropyl acetate) and mixtures thereof.

“Aqueous solvent” is taken to mean a solvent selected from the group consisting of water alone and the combination of water with one or more C1 to C5 alcohols (for example ethanol), one or more C3 to C5 glycols or a mixture thereof.

“Hydro-glycolic solvent” is taken to mean a mixture of water and one or more C3 to C5 glycols, notably propylene glycol, butylene glycol, pentylene glycol, propan-1,3-diol or a combination thereof.

“Dry extract” is taken to mean, in the sense of the present invention, an extract without extraction solvent or support or containing only non-significant trace amounts thereof. Such a dry extract thus only contains material derived from Tahiti vanilla pods. It may also contain non-significant traces of extraction solvent.

“Topical application” is taken to mean an application on the skin, the mucous membranes and/or the skin appendages, preferably the skin.

In the present invention, “cosmetically or dermatologically acceptable” is taken to designate what is useful in the preparation of a cosmetic or dermatological composition, which is generally safe, non-toxic and neither biologically or otherwise non-desirable and which is acceptable for cosmetic or dermatological use, notably by topical application.

“Cosmetically or dermatologically acceptable vehicle” is taken to mean any adjuvant or excipient enabling the production, the conservation and/or the administration of a cosmetic or dermatological composition.

### DETAILED DESCRIPTION

According to a first aspect, the invention relates to a combination of a Tahiti vanilla extract and 4-[(1E,3S)-3-ethenyl-3,7-dimethylocta-1,6-dienyl]phenol, notably for the use thereof for combatting cutaneous ageing.

The Tahiti vanilla plant, Vanilla tahitensis, belongs to the Orchid family. One of the botanic particularities of the genus Vanilla is that it is the only orchid to bear fruit. Tahiti vanilla is one vanilla species among the 119 species inventoried to date.

Vanilla tahitensis is a perennial climbing creeper of green colour, cylindrical, of diameter varying between 1 to 1.5 cm, which in general reaches a height of 15 to 20 metres. Just like ivy, Vanilla tahitensis requires a stake to develop: it is thus an epiphytic species (growing on another plant without being a parasite to it). The gnarled, fleshy stems cling to the support by adventitious roots. The latter develop at the level of nodes and their clinging role has earned them the nickname “claspers”. These aerial roots have in fact a double role: they attach the creeper to the stake and they absorb humidity from the air thanks to their special structure. The green leaves are long and persistent. Some 5 to 20 cm long and 5 to 8 cm wide, they begin at the level of the nodes. At the axil of each leaf there is an eye that can give rise to a secondary ramification. The flowers are large: their diameter can sometimes reach 10 cm. Of light yellow colour, they are grouped together in bunches or in auxiliary spikes grouping together sometimes thirty or so flowers. Each flower is composed of 3 petaloid sepals and 3 petals of which one is more developed than the two others. Its edges are welded together to form a cornet known as the “labellum”. This completely surrounds the sexual organs composed of a stamen constituted of a filament bearing a pollinium and a style surmounted by two fertile stigmas. A quite thick tongue separates the pollinium and the stigmas: it is the “rostellum” that prevents any natural fecundation. To bear fruit, vanilla thus has need of an external agent. Once pollinated, the inflorescence bears a certain number of fruits: it is the “broom”. The fruit is constituted of a capsule, the pod, enclosing 3 loculi and a large number of blackish seeds. The green fruits have a length varying from 10 to 20 cm and a width ranging from 1 to 1.5 cm. The pod is fixed on the “broom” by a “hook”. One of the particularities of Tahitian vanilla compared to its fellow plants lies at the level of its ripening. Whereas other species of vanilla absolutely have to be harvested before maturity, at the risk if not of seeing their pods burst, Vanilla tahitensis is harvested without damage at maturity, while standing.

Several elements are prerequisites for the culture of Vanilla tahitensis. The latter has in fact very marked climatic requirements: the soil must be light, drainable and rich in humus and mineral matter. Abundant rainfall is indispensable. Vanilla plants have to be cultivated at an altitude below 600 metres, under a soft light often obtained by shading. Finally, the ideal temperature is comprised between 20 and 30° C. The vanilla plant propagates easily from cuttings, in several weeks it has developed its roots. At the end of 3 years the first flowering takes place.

The fruit develops to reach 15 to 20 cm in several months. When it reaches its definitive size, the fruit becomes a brilliant green identical to that of the stems and the leaves. It releases no odour at this point. At the end of 8 to 9 months, the fruit becomes pale green, then turns to yellow and finally to brown at its end, it is the signal for the harvest. This is done by hand, via a slight torsion. Once collected, the pods are kept in the dark for several days and become uniformly brown. Next, the pods are dried 2 hours a day by the sun. At least two months are necessary to obtain black pods of wilted appearance. It is during this drying period that the vanilla acquires its aroma recognisable by all. Tahiti vanilla has a particular aroma linked to its phytochemistry: heliotropin, a highly odorous and heady molecule, is in fact only found in Vanilla tahitensis.

According to the bibliography, Vanilla tahitensis mainly contains:
polyphenols and polyphenolic derivatives: heliotropin, vanillin, anisic alcohol, anisic acid, parahydroxybenzoic acid, etc.;
sugars;
fatty matter;
minerals.

According to an embodiment of the invention, the Tahiti vanilla extract is a hydrophilic extract, more specifically an aqueous extract, even more specifically a hydro-glycolic extract.

This extract could notably be obtained by extraction with at least one hydrophilic solvent, preferentially with at least one aqueous solvent and even more preferentially with at least one hydro-glycolic solvent.

According to a particular embodiment, this extract is obtained by extraction of Tahiti vanilla pods, using one or more hydro-glycolic solvent(s), such as a water/propylene glycol mixture, for example in a water/propylene glycol ratio of 25/75 to 40/60.

The extract obtained may be used as is or in concentrated extract or dry extract form after partial or total evaporation of the extraction solvent.

The Tahiti vanilla extracts used in the compositions according to the invention will advantageously include a dry extract content of 0.5% to 5%, preferably 0.6 to 2% by weight compared to the total weight of the extract.

4-[(1E,3S)-3-ethenyl-3,7-dimethylocta-1,6 dienyl]phenol or Bakuchiol is a highly purified meroterpene compound, described in the U.S. Pat. No. 8,529,967, known under the CAS number: 10309-37-2. Meroterpenes are generally obtained from plants or plant extracts. They may also be obtained from mushrooms or instead be synthesised. Plants serving as sources for meroterpenes are for example Psoralea coryfolia, Psoralea grandulosa and Otholobium pubescens.

In practice, the meroterpene may be supplied by the addition of isolated and purified meroterpene, preferably having a degree of purity at least equal to 60%, or even at least 70%, 80%, 90% or even at least equal to 95%, or by the addition of a plant extract including meroterpene.

Advantageously, the bakuchiol is supplied by the addition of a plant extract including it, preferably an extract of Psoralea coryfolia seeds.

According to another embodiment, the bakuchiol is isolated and purified from the plant Psoralea coryfolia, advantageously from its seeds, such as for example the titrated bakuchiol product, sold by the SYTHEON LTD Company under the denomination Sytenol®. In this product the bakuchiol has a purity greater than or equal to 95%.

The present invention thereby relates to a combination including a Tahiti vanilla extract and bakuchiol.

According to a particular embodiment, the invention relates to a combination including an aqueous extract of Tahiti vanilla and bakuchiol.

According to a particular embodiment, the invention relates to a combination including a hydro-glycolic extract of Tahiti vanilla and bakuchiol.

According to another particular embodiment of the invention, the latter relates to a combination according to the invention according to any one of the embodiments described previously for the use thereof intended to combat signs of cutaneous ageing, more specifically to combat photo-induced cutaneous ageing. Preferably this combination is used by topical route.

According to another aspect, the invention relates to a cosmetic use of the combination according to the invention according to any of the embodiments described previously for limiting cutaneous ageing, and/or preventing and/or reducing wrinkles, and/or toning up the skin, and/or relaunching epidermal and dermal cellular activity.

According to another aspect, the invention relates to a use of the combination according to the invention according to any of the embodiments described previously for the manufacture of a cosmetic or dermatological composition, preferably topical, intended for combating cutaneous ageing, photo-induced or not, and more particularly intended for limiting cutaneous ageing, and/or preventing and/or reducing wrinkles, and/or toning up the skin, and/or relaunching epidermal and dermal cellular activity.

According to another aspect, the invention relates to a cosmetic method for combating cutaneous ageing, photo-induced or not, and more particularly intended for limiting cutaneous ageing, and/or preventing and/or reducing wrinkles, and/or toning up the skin, and/or relaunching epidermal and dermal cellular activity, comprising the administration, preferably by topical application, in particular on the skin, to a person in need thereof of a combination according to the invention according to any of the embodiments described previously.

The present invention also relates to a cosmetic or dermatological composition including as active ingredients a Tahiti vanilla extract and bakuchiol, and at least one cosmetically or dermatologically acceptable vehicle.

According to a preferred embodiment, the cosmetically or dermatologically acceptable vehicle comes in a form intended for topical application. The subject matter of the invention is quite particularly intended for topical application on cutaneous zones considered as fragile, such as for example the face, the neck, the contour of the eyes and the lips. Any vehicle acceptable from the cosmetic or dermatological viewpoint, selected for example from adjuvants and excipients commonly used in galenics, may be used in the composition according to the invention.

The cosmetic or dermatological compositions according to the invention could come in forms that are normally known for topical administration, that is to say notably lotions, foams, gels, dispersions, emulsions, sprays, serums, masks, creams, or jellies, notably micellar jellies, with excipients notably enabling better bioavailability in order to improve the properties and the accessibility of the active ingredients. Advantageously, it is a cream, a balm, or a serum.

These compositions generally contain, apart from the compounds of the combination according to the present invention, a physiologically acceptable medium, in general based on water or solvents, for example alcohols, ethers or glycols. They can also contain surfactants, complexing agents, preservatives, stabilisers, emulsifiers, thickeners, gelling agents, humectants, emollients, trace elements, essential oils, fragrances, colorants, matting agents, chemical or mineral filters, hydrating agents, thermal waters, etc.

Advantageously, the compositions according to the present invention will include 0.01% to 10% by weight, preferably 0.1% to 5% by weight, in a preferred manner 0.2% to 2% by weight, in an even more preferred manner 0.3% to 1.5% by weight of Vanilla tahitensis extract, compared to the total weight of the composition. In a preferred manner, the composition will include around 0.5% by weight of Vanilla tahitensis extract, compared to the total weight of the composition. In an equally preferred manner, the composition will include around 1% by weight of Vanilla tahitensis extract, compared to the total weight of the composition.

According to another embodiment, the compositions according to the present invention will include 0.00005 to 0.5%, preferably 0.0005 to 0.25%, in an even more preferred manner 0.001 to 0.1%, in a yet even more preferred manner 0.0015 to 0.075% of Tahiti vanilla extract by weight of dry extract compared to the total weight of the composition.

Advantageously, the compositions according to the present invention will include 0.01% to 10% by weight, preferably 0.1% to 5% by weight, in a preferred manner 0.2% to 3% by weight, in an even more preferred manner 0.5% to 2% by weight of bakuchiol, compared to the total weight of the composition. In a preferred manner, the composition will include around 1% by weight of bakuchiol, compared to the total weight of the composition. In an equally preferred manner, the composition will include around 1.5% of bakuchiol, compared to the total weight of the composition.

In a particular manner, the invention relates to a composition in which the Tahiti vanilla extract:bakuchiol weight ratio is comprised between 1:15 and 10:1, preferentially between 1:10 and 5:1.

The compositions according to the invention may be used in cosmetology and in dermatology to prevent or improve wrinkled skin and make it possible to maintain the firmness and the moisture of the skin.

The compositions according to the invention may be used in topical preparations, in the field of dermatology or cosmetology, with the aim of preventing and/or reducing wrinkles, combating cutaneous ageing whether photo-induced or not, toning up the skin, relaunching epidermal and dermal cellular activity.

The invention also relates to the cosmetic use of the composition according to the invention for combatting ageing of the skin, and more specifically combatting photo-induced cutaneous ageing.

The invention also relates to a cosmetic method for combatting cutaneous ageing whether photo-induced or not, including the application on the skin of the composition according to the invention.

Such compositions may be manufactured according to methods well known to those skilled in the art.

The following examples illustrate the invention without limiting the scope thereof.

### Example 1: Anti-Ageing Effect of a Vanilla Extract, Bakuchiol and the Combination Thereof

The ageing of organic tissues, and in particular that of the skin, is a natural and complex biological process. It is composed of two types of ageing: chronological or intrinsic ageing which is an inevitable process mainly linked to age, and extrinsic ageing linked to environmental factors such as UVs responsible for premature ageing of the skin called photo-ageing. One of the causes that explains in part the functional decline of the skin with age is that the regeneration capacity of the cells, essential for tissue functions, is altered over time. However, it has recently been suggested that ageing is also associated with the accumulation of so-called senescent cells which have a specific phenotype: they secrete cytokines and other mediators which influence the working of the skin (secretome) and are incapable of dividing in the tissue (stoppage of the cellular cycle). The cellular cycle is a complex mechanism which is thus strongly influenced by ageing. Among the molecular markers of the senescence process, the protein P16 is a specific inhibitor of the cellular cycle which brings about a blockage of cellular proliferation in G1 phase and thereby participates in the induction of the senescence programme responsible for cell ageing.

The aim of this study is to evaluate the effects of bakuchiol, a Tahiti vanilla extract and the combination thereof on the prevention of ageing after acute stress to UVA on normal human fibroblasts.

The biological model used for this study consists of normal human dermis fibroblasts obtained from healthy donors.

The fibroblasts are cultured in a standard culture medium for 36 hours, the products to test (vehicle (DMSO), bakuchiol (0.5 μg/ml), vanilla extract (0.05%, p/v) and the combination thereof (same concentrations)) are added to the culture medium one hour before the UVA radiation test. This test consists in a photo-ageing stress: the fibroblasts are irradiated by UVA at a dose of 60 J/cm² (Waldmann UVA lamp). A condition without irradiation is also realised. The fibroblasts are next sampled 24 h after irradiation for immunofluorescence analyses. All the experimental conditions are realised with N=3 donors. The fluorescence was quantified using Nikon NIS Element software in order to obtain an average fluorescence per cell and per condition.

The Tahiti vanilla extract tested in this study was obtained from the hydro-glycolic extraction of Tahiti vanilla pods, using a water/propylene glycol mixture as extraction solvent. This extract includes around 64.7% of propylene glycol, 34.5% of water and around 0.8% of dry extract. It is a commercially available material Vanirea® UP sold by the Solabia Company. The INCI name of this material is: propylene glycol/water/Vanilla tahitensis. The CAS number of Vanilla tahitensis is: 94167-14-3.

The bakuchiol tested in this study corresponds to the commercially available material Sytenol® UP of the supplier Sytheon. Its CAS number is 10309-37-2.

The treatment of human fibroblasts with acute UVA stress induces a significant increase in the expression of P16, going from 500 AU to almost 1000 AU, P<0.001, versus the non-stimulated condition. This result makes it possible to validate the test. The statistics are produced using a one way variance analysis (one-way ANOVA) followed by a Dunnett test.

The protein P16 is normally weakly expressed in the cells and its expression is increased during stress or ageing to induce a stoppage of the cellular cycle and favour the activation of the senescence programme in the cells. Furthermore, searching for the expression of the protein P16 by immunohistochemistry or immunocytochemistry is a means for establishing the diagnosis of the presence of senescent cells in the tissues.

The treatment of fibroblasts with the vanilla extract tested at 0.05% tends to decrease the expression of P16 strongly induced by UVA stress; this decrease is only 29.2%, P═NS versus the non-treated group. Similarly, the treatment of fibroblasts by bakuchiol tested at 0.5 μg/mL reduces the expression of P16 following irradiation, but this reduction does not reach significance, −44.4%, P═NS versus the non-treated group. On the other hand, the treatment of fibroblasts by the combination of these 2 compounds, at the same concentrations, enables a significant prevention of the increase in the expression of P16 induced by UVA stress, this reduction reaching 95.2%, P<0.001 versus the non-treated group.

The compounds alone cannot prevent the expression of P16 in a significant manner. However, the addition of their own effects (29.2+44.4=73.6% protection) is well below the protection brought about by their combination (95.2>>73.6% protection). The Inventors thereby demonstrate a synergy of action by combining a vanilla extract and bakuchiol.

These results make it possible to demonstrate that the combination of bakuchiol and a vanilla extract prevents the senescence of cells linked to UVA. By acting on the protein P16, this combination is going to decrease the activation of the senescence programme in the cells and thus make it possible to relaunch the activity of epidermal and dermal cells. The Inventors thereby demonstrate that this combination is thus provided with anti-ageing properties.

### Example 2: Anti-Inflammatory Effect of a Vanilla Extract, Bakuchiol and the Combination Thereof

Ageing is strongly linked to an alteration of the immune and inflammation system. For example, the aged dermis accommodates more mastocytes and neutrophils. This phenomenon is correlated with an up-regulation of interleukin 8 (IL8) in aged skin. IL8 is a pro-inflammatory and chemotactic cytokine. Even though the main source of IL8 is monocytes, it is also produced by macrophages, endothelial cells, keratinocytes and fibroblasts. On account of their location, the production of IL8 by fibroblasts can play an important role in communication with the endothelial cells of the dermis, increasing the migration of neutrophils into the dermis and accentuating inflammation. In addition, the synthesis of IL8 by the fibroblasts of the dermis is considerably influenced by UV irradiation.

The aim of this study is to evaluate the effects of bakuchiol, a vanilla extract and the combination thereof on the prevention of inflammation, notably on the production of IL8 after acute UVA stress on normal human fibroblasts.

The protocol used is rigorously the same as that described in example 1. The products tested are the same, the concentrations also. The expression of IL8 is then quantified by immunofluorescence.

The treatment of human fibroblasts with acute UVA stress induces a significant increase in the expression of IL8, going from 100% to 5300% (P<0.05) compared to the condition without irradiation. In addition, it appears that the treatment by UVA induces important morphological changes in the fibroblasts, going from a star-shaped model to a spindle-shaped model. These results make it possible to correctly validate this model.

The treatments of fibroblasts with the vanilla extract or bakuchiol or the combination thereof result in a prevention of the morphological changes induced by exposure to UVA, which indicates a potential activity of these compounds.

The treatment of human fibroblasts with the vanilla extract, tested at 0.05% (p/v) results in an evident tendency to prevent the expression of IL8 induced by stress to irradiation, with an inhibition of 83.8%, (P═NS versus non-treated conditions). With bakuchiol (0.5 μg/ml), the expression of IL8 following exposure to UVA is significantly decreased (88.3%, P<0.05 versus non-treated conditions). The treatment of fibroblasts by the combination of these 2 compounds at the same concentrations enables a significant prevention of the increase in the expression of IL8 induced by UVA stress, this reduction reaches 95.1%, P<0.05 versus the non-treated group.

Thus, the Inventors demonstrate that the combination of a vanilla extract and bakuchiol induces a more important anti-inflammatory effect than that of the two compounds taken in isolation.

### Example 3: Examples of Compositions Including Bakuchiol and a Tahiti Vanilla Extract According to the Invention

### Composition for a Day Cream:

| | Percentage by |
|---|---|
| INCI designation | weight |
| Water | QS 100% |
| Glycerine | 5.0 |
| Disodium EDTA | 0.1 |
| Hexanediol | 0.5 |
| Polyacrylate | 1.2 |
| Behenic alcohol | 1.0 |
| Caprylic/Capric triglycerides | 6.0 |
| Dicaprylyl carbonate | 7.0 |
| Capryl glycol | 0.5 |
| Beeswax | 1.0 |
| Tribehenin PEG-20 esters | 5.0 |
| Sytenol | 1.0 |
| Vanilla extract | 0.5 |
| Fragrance | 0.2 |

### Composition for a Night Cream:

| | Percentage by |
|---|---|
| INCI designation | weight |
| Water | QS 100% |
| Glycerine | 10.0 |
| Disodium EDTA | 0.1 |
| Hexanediol | 0.5 |
| Polyacrylate | 1.2 |
| Behenic alcohol | 2.0 |
| Caprylic/Capric triglycerides | 10.0 |
| Dicaprylyl carbonate | 7.0 |
| Capryl glycol | 0.5 |
| Beeswax | 1.0 |
| Ceterayl glucoside & Cetearyl | 2.0 |
| alcohol | |
| Glyceryl stearate & PEG-100 | 3.0 |
| stearate | |
| Sytenol | 1.0 |
| Vanilla extract | 0.5 |
| Fragrance | 0.2 |

### Composition for a Serum:

| | Percentage by |
|---|---|
| INCI designation | weight |
| Water | QS 100% |
| Glycerine | 10.0 |
| Disodium EDTA | 0.1 |
| Hexanediol | 0.5 |
| Polyacrylate | 1.2 |
| Cetyl alcohol | 1.5 |
| Caprylic/Capric triglycerides | 10.0 |
| Dicaprylyl carbonate | 3.0 |
| Capryl glycol | 0.5 |
| Sytenol | 1.5 |
| Vanilla extract | 1.0 |
| Fragrance | 0.2 |

1. A combination including 4-[(1E,3S)-3-ethenyl-3,7-dimethylocta-1,6-dienyl]phenol and a Tahiti vanilla pod extract.

2. The combination according to claim 1, wherein the Tahiti vanilla pod extract is a hydro-glycolic extract.

3.-5. (canceled)

6. A cosmetic or dermatological composition including, as active ingredient, a combination according to claim 1, with at least one cosmetically or dermatologically acceptable vehicle.

7. The composition according to claim 6, including 0.01% to 10% by weight of a Tahiti vanilla pod extract compared to the total weight of the composition.

8. The composition according to claim 6, including 0.01% to 10% by weight of 4-[(1E,3S)-3-ethenyl-3,7-dimethylocta-1,6-dienyl]phenol compared to the total weight of the composition.

9. The composition according to claim 6, being in a form suitable for topical application.

10.-12. (canceled)

13. The composition according to claim 6, including 0 0.1% to 5% by weight of a Tahiti vanilla pod extract compared to the total weight of the composition.

14. The composition according to claim 6, including 0.2% to 2% by weight of a Tahiti vanilla pod extract compared to the total weight of the composition.

15. The composition according to claim 6, including 0.1% to 5% by weight of 4-[(1E,3S)-3-ethenyl-3,7-dimethylocta-1,6-dienyl]phenol compared to the total weight of the composition.

16. The composition according to claim 6, including 0.2% to 3% by weight of 4-[(1E,3S)-3-ethenyl-3,7-dimethylocta-1,6-dienyl]phenol compared to the total weight of the composition.

17. A method for combating cutaneous ageing comprising the administration to a person in need thereof of a combination according to claim 1.

18. The method according to claim 17, wherein the cutaneous ageing is a photo-induced cutaneous ageing.

19. A cosmetic method for limiting cutaneous ageing, and/or preventing and/or reducing wrinkles, and/or toning up the skin, and/or relaunching epidermal and dermal cellular activity comprising the administration to a person in need thereof of a combination according to claim 1.

20. A method for combating cutaneous ageing comprising the administration to a person in need thereof of a composition according to claim 6.

21. The method according to claim 20, wherein the cutaneous ageing is a photo-induced cutaneous ageing.

22. A cosmetic method for limiting cutaneous ageing, and/or preventing and/or reducing wrinkles, and/or toning up the skin, and/or relaunching epidermal and dermal cellular activity comprising the administration to a person in need thereof of a composition according to claim 6.
